(19) United States
(12) Patent Application Publication
(10) Pub. No.: US 2010/0268160 A1
(43) Pub. Date: Oct. 21, 2010

(54) DISPOSABLE SYRINGE WITH PROTECTION AGAINST REUSE

(75) Inventor: Monika Eccard, Kernen (DE)

Correspondence Address: Brinks Hofer Gilson & Lione/Ann Arbor, 524 South Main Street, Suite 200, Ann Arbor, MI, 48104, US

(73) Assignee: CET Consulting Engineering Trading Monika Eccard, Kemen (DE)

(21) Appl. No.: 12/631,572

(22) Filed: Dec. 4, 2009

Related U.S. Application Data
(63) Continuation of application No. PCT/EP2008/004441, filed on Jun. 4, 2008.

(30) Foreign Application Priority Data
Jun. 4, 2007 (DE) 10 2007 026 972.4

(51) Int. Cl. A61M 5/50

(52) U.S. Cl. 604/110

(57) ABSTRACT

A disposable syringe with protection against reuse is proved. The syringe includes a cylinder with an injection nozzle and a piston that has a piston section facing the injection nozzle, a piston rod and an actuation section facing away from the piston section, wherein the piston rod has a latching section with a shoulder that is provided in or on the piston rod and runs radially inward, the cylinder has a counter-latching section protruding inward, and the piston has at least one predetermined breaking point in such a way that when the piston rod is introduced into the cylinder the counter-latching section engages behind the shoulder to lock it and counteracts removal the piston rod from the cylinder, and wherein the predetermined breaking point is designed such that it breaks when pulling on the actuation section.

### CROSS-REFERENCE TO RELATED APPLICATIONS

This application is a continuation of International Application No. PCT/EP2008/004441 filed on Jun. 4, 2008, which claims the benefit of DE 10 2007 026 972.4, filed Jun. 4, 2007. The disclosures of the above applications are incorporated herein by reference.

### FIELD

The disclosure relates to a disposable syringe with protection against reuse.

### BACKGROUND

The statements in this section merely provide background information related to the present disclosure and may not constitute prior art.

Disposable syringes are known from EP 557 511 A1, GB 2 246 297 A, and EP 442 260 A1, which have latching sections in the area of the injection nozzle and the side of the piston section facing the injection nozzle, said latching sections locking with each other when completely introducing the piston section into the cylinder. A predetermined breaking point, which is likewise present in this front area of the disposable syringe, ensures reliable destruction of the syringe after a single use. The disadvantage of these disposable syringes is that the latching sections are located in an area of the syringe to be filled with the substance to be injected and thus upon use of the syringe are exposed to direct contact with the substance to be injected. In addition, the latching sections limit the usable volume of the cylinder. Furthermore, the latching sections of the syringes described in the first two documents named include hollow spaces, which form a blind volume that receives part of the substance when drawing up the substance to be injected into the syringe, however this part remains in the syringe during the subsequent injection.

A syringe is known from US 2004/0127859 A1 that has latching sections that during the injection process do not come in contact with the substance to be injected.

### SUMMARY

The present disclosure provides a disposable syringe with protection against reuse, which can be produced simply and at a favorable cost and the means of which for offering protection against reuse are disposed outside the area of the disposable syringe to be filled with the substance to be injected.

As a result of a latching section, a counter-latching section, and a predetermined breaking point being located outside of the disposable syringe area to be filled with a fluid to be injected, they take up no volume of the fillable area, whereby a relatively short cylinder can be provided, especially for disposable syringes having small diameters, and, for example, a printed scale to indicate the current volume of the area to be filled can be linearly designed on the cylinder. In this way, the use of this disposable syringe with protection against reuse is made easier. In addition, the disposable syringe has no blind volume caused by the reuse protection and no narrowing in the area of the injection nozzle caused by a latching section or a predetermined break point.

It is especially preferred that the latching section comprises a shoulder that is provided in the piston rod and runs radially inward, and that the counter-latching section is designed to protrude inward and engages behind the shoulder so as to lock it when the piston rod is introduced, wherein the counter-latching section, at least during a section of the insertion path before complete introduction of the piston into the cylinder, rests against the piston rod under pretension in such a way that a force acting against the introduction in the axial direction along the entire insertion path only increases slightly. This at least largely prevents the force acting against the introduction, which the user of the disposable syringe perceives as resistance when introducing the piston into the cylinder, from increasing abruptly when the latching section passes the counter-latching section. As a result, simple and user-friendly handling of the disposable syringe is assured.

The disposable syringe can advantageously be designed such that a first component of the insertion force, which is caused by the interaction of the latching section with the counter-latching section, preferably along the entire insertion path, is significantly smaller than a second component of the insertion force, which is caused by friction of the piston section on an interior wall of the cylinder. The influence of the latching section or the counter-latching section on the overall force acting against the introduction is thereby reduced. A relative change of the force because of the interaction of the latching section with the counter-latching section is thus reduced, and handling of the disposable syringe is further facilitated.

In one form, the counter-latching section rests under pretension against the piston rod for at least almost the entire insertion path of the piston. To this end, it may be provided that on the side of the shoulder facing the piston section, the piston comprises a section which is bounded radially to the outside in a longitudinal sectional view by edges running parallel to a central longitudinal axis of the piston.

It can also be provided that the latching section rests under pretension against the counter-latching section during a section of the insertion path of the piston, wherein in order to produce the pretension the latching section comprises a chamfer which is bounded by the shoulder on a side facing the actuation section, wherein the chamfer has a length extending in the axial direction which amounts to at least 1.5 times, preferably at least five times, and further preferred at least ten to twenty times, the height of the chamfer extending in a radial direction. This produces a relatively slight incline of the chamfer compared to the central longitudinal axis of the piston, which results in a comparatively small first component of the force caused by the interaction of the latching section with the counter-latching section. The height of the chamfer corresponds to a distance between a base of the preferably substantially triangular sectional plane of the chamfer and the edge of the shoulder facing away from the central longitudinal axis. It may be provided that the height of the chamfer corresponds to the width of the shoulder. The chamfer causes in particular the first component of the force caused by the interaction of the latching section with the counter-latching section to increase slowly and continuously during the introduction of the piston into the cylinder, without making the handling of the disposable syringe more difficult. The chamfer can be designed such that it deforms elastically when it is under pretension. Once the chamfer passes the counter-latching section, the pretension is basically eliminated, the chamfer again largely assumes the original shape thereof, and the counter-latching section surrounds the shoulder.

It is conceivable that the length of the chamfer corresponds at least to one tenth of the length of the piston rod with the piston section.

Furthermore it is advantageous if the counter-latching section is provided in a cylinder section having a larger inside diameter than a main cylinder section of the cylinder, wherein the main cylinder section is used in particular to receive the serum to be injected. As a result, the narrow point of the counter-latching section can be designed to protrude radially inward such that it has a diameter which corresponds approximately to the diameter of the remaining cylinder or it can also be slightly greater. This results in simplified handling of the syringe and manufacturing advantages.

It may also be provided that the counter-latching section is designed resilient in the radial direction in at least some sections so as to produce the pretension.

To this end, it is preferred that the counter-latching section comprises at least one flexible tongue with a partial section protruding radially inward and interacting with the shoulder in order to produce the pretension, wherein the flexible tongue is disposed in particular in the axial direction so as to protrude on the side of the cylinder facing away from the injection nozzle. By using the flexible tongue, an especially elastic counter-latching section can be implemented, which, as long as it is not locked, presses against the piston rod with only a slight force, but in the locked state engages tightly in the shoulder of the piston rod and thus acts against a removal the piston rod. This enables the production of an especially smoothly moving syringe with a highly effective latching mechanism.

It is furthermore conceivable that the latching section and/or the chamfer of the latching section is designed resilient in the radial direction and/or a direction that is transverse to the longitudinal direction in order to produce the pretension.

It may also be provided that the latching section at least in some sections comprises a circumferential, radially outwardly directed annular collar and the counter-latching section at least in some sections comprises a narrow point directed radially inward, wherein the maximum outside diameter of the annular collar is larger than the minimum inside diameter of the narrow point and the narrow point of the counter-latching sections can be elastically expanded at least to a limited degree such that the annular collar can pass the narrow point upon insertion of the piston rod into the cylinder. Both the latching section and the counter-latching section can be manufactured very simply using methods that are customarily used in the production of plastic parts. The annular collar can be designed in a simple manner as a regional thickening of the piston rod, while the narrow point can be designed in an equally simple manner by a regional thickening of the cylinder wall of the syringe. In order to increase the elasticity of the narrow point, it may also be provided that the cylinder includes notches, thin spots or the like.

It may be provided that the latching section on the side thereof facing the piston section, and/or that the counter-latching section on the side thereof facing away from the injection nozzle, comprise at least one stop surface which is disposed approximately or exactly orthogonally to a center longitudinal axis of the cylinder. In this way, as soon as the latching section has passed the counter-latching section during the introduction of the piston rod into the cylinder, renewed passage of the latching section is not possible, or is only possible with very forceful pulling on the piston rod. As a result, the removal of the piston rod following the introduction is counteracted.

It may furthermore be provided that the latching section on the side thereof facing the piston section, and/or that the counter-latching section on the side thereof facing away from the injection nozzle, comprise an insertion taper. In this way, the latching section can pass the counter-latching section during introduction of the piston rod into the cylinder with relatively light pressure on the piston rod. Pushing the annular collar forward into the narrow point is also facilitated. Thus, resistance that may be higher in some areas when handling the syringe during an injection process is reduced when pushing in the piston rod.

It may also be provided that the predetermined breaking point is located between the piston rod and the actuation section. As a result, when pulling on the actuation section with a locked latching section and counter-latching section for the purpose of pulling out the piston rod, exactly this component of the syringe, namely the actuation section, which must be present to impart the tensile force required to overcome the retaining force of the latching section and the counter-latching section, is detached from the remaining syringe. The syringe is thereby rendered unusable and any attempt at renewed use of the syringe is thwarted.

It may be provided that the predetermined break point comprises a thinned area and/or an area having at least one transverse hole. In this way, the predetermined breaking point can be implemented in a particularly simple and cost-effective manner using customary methods for producing plastic parts.

It is particularly preferred that the piston rod and/or the actuation section have contact sections that interact with each other and are designed separately from the predetermined breaking point in such a way that bending or breaking of the predetermined breaking point while pressing in the piston is prevented. As a result of the contact sections resting on top of each other when applying pressure on the actuation section for the purpose of introducing the piston rod, and thus absorbing at least part of the pressure force and/or laterally acting forces, the predetermined breaking point is stabilized upon introduction of the piston into the cylinder, while it easily breaks under a tensile stress, because the contact sections cannot mutually interact under tensile stress.

It is particularly preferred that the contact sections have a surface running exactly or approximately perpendicularly and/or parallel to the center longitudinal axis of the piston. In the first case, the contact sections can substantially absorb the pressure forces acting in the longitudinal direction of the piston rod, while in the latter case it can absorb transverse forces acting substantially perpendicularly to the piston rod which may appear upon actuation of the syringe, for example, during an injection. Furthermore, interfering back and forth movements of the actuation section that occur during the actuation of the syringe and result, for example, from the elasticity of the predetermined breaking point are avoided.

It may also be provided that the contact sections of the piston rod and actuation section are designed conically and run at least substantially parallel to each other. As a result, the contacts can be implemented in a particularly simple manner using customary methods for producing plastic parts.

It is particularly preferred that the contact sections are provided in the area of the predetermined breaking point. Contact sections designed in this way are especially effective and do not interfere in the handling of the disposable syringe. They can, however, be implemented in a simple manner.

In order to attain predetermined breaking points which can be produced with relatively little effort using customary method for producing plastic parts, it is also recommended that the piston comprise at least two predetermined breaking points designed as connection surfaces, which are disposed at a distance from each other, preferably within a plane running orthogonally to the central longitudinal axis of the piston. This additionally provides good strength of a connection between the actuation section and the piston rod with laterally acting forces acting.

In order to further stabilize the connection between the actuation section and the piston rod in such a way that it can absorb laterally acting forces, it is proposed that the piston comprise a predetermined breaking point designed as a center connection surface in a center area of a cross-sectional surface of the piston rod, wherein the center connection surface is preferably disposed within the plane running orthogonally to the center longitudinal axis of the piston.

Furthermore, it may be provided that a distance between the predetermined breaking points amounts to at least half of the radius of the piston rod.

According to one form of the present disclosure, the cross-section piston rod and/or the actuation section, at least in some sections, may be composed of several, preferably four, flat segments protruding radially from the center longitudinal axis. It may be provided that all adjacent segments are inclined toward each other at the same angle. In the case of four segments, this results in a cross-shaped cross-section of the piston rod or the actuation section, wherein the individual segments of the cross are disposed at an angle of 90 degrees. The segments may comprise the latching sections and in particular, if provided, the associated chamfers.

It is particularly preferred that the piston section, the piston rod, the actuation section, and the latching section are connected to each other as a single piece and/or the cylinder and the counter-latching section are connected to each other as a single piece. In this way, such disposable syringes can be provided, for example, which are composed of only two parts. Because of this small number of parts, the syringe can be produced in a particularly simple manner, quickly and using fewer work steps, and thus more cost effectively.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

Further advantageous forms and details of the invention are apparent from the following description, which describes and explains the present disclosure in more detail based on the various forms depicted in drawings.

FIG. 1: A cut side view of a claimed disposable syringe according to a first form of the present disclosure with a partially introduced piston;

FIG. 2: A cut side view of the disposable syringe from FIG. 1 with a completely introduced piston;

FIG. 3: A cut side view of a piston of a claimed disposable syringe according to a second form of the present disclosure;

FIG. 4: A cut side view of a disposable syringe according to a third form of the present disclosure with an incompletely introduced piston;

FIG. 5: A top view of a cut-out of a cylinder of a claimed disposable syringe according to a fourth form of the present disclosure;

FIG. 6: A cut side view of a cut-out of a piston of a claimed disposable syringe according to a fifth form of the present disclosure;

FIG. 7: A cross-section of the piston shown in FIG. 6 along a cut line shown in FIG. 6;

FIG. 8: A cut side view of a cut-out of a piston of a claimed disposable syringe according to a sixth form of the present disclosure;

FIG. 9: A cross-section of the piston shown in FIG. 8 along the cut line shown in FIG. 8;

FIG. 10: A cut side view of a cut-out of a piston of a claimed disposable syringe according to a seventh form of the present disclosure;

FIG. 11: A cut side view of a cylinder of a claimed disposable syringe according to an eighth form of the present disclosure;

FIG. 12: A cut side view of a piston of the disposable syringe from FIG. 11;

FIG. 13: A cross-section of the piston shown in FIG. 12 along the cut line shown in FIG. 12;

FIG. 14: A cut side view of a piston of the disposable syringe shown in FIGS. 11-13 with a piston completely introduced into the cylinder; and

FIG. 15: A cross-section of a piston of a disposable syringe similar to FIG. 13 according to a ninth form of the present disclosure.

In the individual figures, identical parts are denoted by the same reference numerals. In general, the parts are described in detail only once.

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

In the individual figures, identical parts are denoted by the same reference numerals. In general, the parts are described in detail only once.

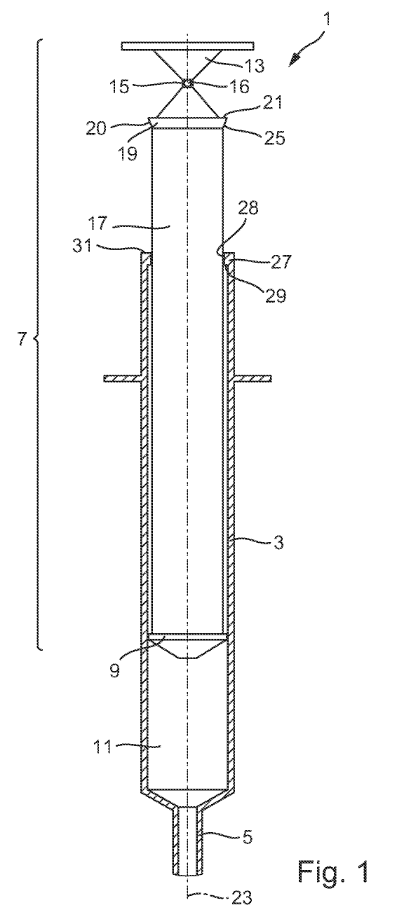

FIG. 1 shows a possible design of a disposable syringe 1 according to the present disclosure, comprising a cylinder 3 which on one side has an injection nozzle 5 and in the side of which facing away from the injection nozzle 5 a piston 7 is introduced. The piston 7 on the side thereof facing away from the injection nozzle has a piston section 9, which together with the remaining piston 7 can be moved back and forth in the cylinder 3. An area which has a variable size and can be filled with a substance to be injected is formed in the cylinder 3 between the injection nozzle 5 and the piston section 9, said area being bounded by the cylinder 3 and the piston section 9. On the side opposite of the piston section 9, the piston 7 has an actuation section 13 which is connected by a predetermined breaking point 15 to the remainder of the piston 7. The predetermined breaking point 15 comprises a transverse hole 16, which is dimensioned such that each area of the cross-section of the predetermined breaking point 15 covered by material from which the predetermined breaking point 15 is made has such a small surface area that the predetermined breaking point breaks when applying a tensile force that meets or exceeds a value of FB of a breaking force.

The piston 7 comprises a piston rod 17 between the predetermined breaking point 15 and the piston section 9, said rod in turn comprising a latching section 19 protruding radially outward. The latching section 19 comprises a circumferential annular collar 20 having an asymmetrical cross-section, which is to say, the latching section 19 on the side thereof facing the actuation section 13 has a stop surface 21 in the form of a planar, ring-shaped surface that is orthogonal to a center longitudinal axis 23 of the piston 7. On the side facing the injection nozzle 5, the latching section 19 further comprises an insertion taper 25, which is formed by a conical, planar surface that is inclined with respect to the center longitudinal axis 23 of the piston 7.

The cylinder 3 on the side thereof facing away from the injection nozzle 5 comprises an inwardly protruding counter-latching section 27 which is complementary to the latching section 19 and bounds a ring-shaped narrow point 28. On the side facing the injection nozzle 5, the counter-latching section 27 comprises a circular-ring shaped, planar counter-stop surface 29, which can interact with the stop surface 21 of the latching section 19. In addition, the counter-latching section 27 on the side thereof facing away from the injection nozzle 5 comprises a circular-shaped, planar delimiting surface 31, which is located orthogonally to the center longitudinal axis 23 of the piston 7. In a form not depicted, instead of the delimiting surface 31 the counter-latching section 27 may alternatively comprise an insertion taper that is complementary to the insertion taper 25 and can be formed similar to the insertion taper 25 by a conical, planar surface that is inclined with respect the center longitudinal axis 23 of the piston 7.

The sequence of use of the disposable syringe 1 is as follows. A disposable syringe 1 issued to a user is in a state in which the piston 7 is introduced as far as possible into the cylinder 3 such that the fillable area 11 has the smallest possible volume and the latching section 19, similar to the state of the disposable syringe 1 shown in FIG. 1, is located on the side of the counter-latching section 27 facing away from the injection nozzle 5. By pulling out the piston rod 17 with the piston section 9, the user can enlarge the fillable area 11 by pulling on the actuation section 13 and draw a substance to be injected up into the syringe 1. In order to preclude a breaking of the predetermined breaking point 15 while drawing up the substance into the disposable syringe 1, the force FB, which would result in breakage of the predetermined breaking point 15 under a tensile stress, is significantly greater than a force Fz which is required to lift the piston section 9.

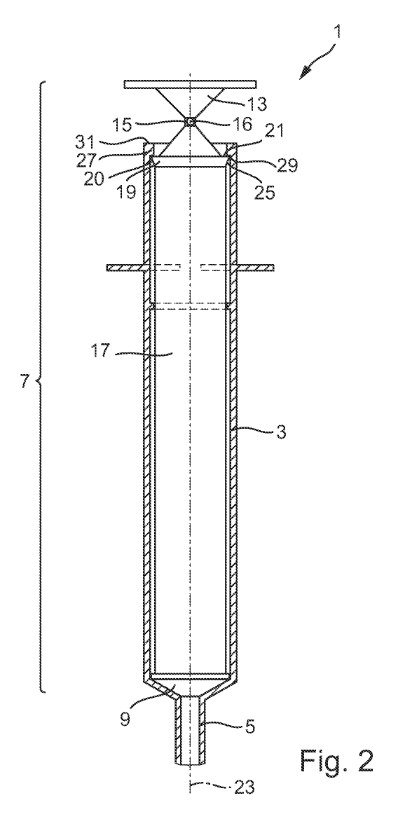

After the filling the fillable area 11 of the disposable syringe 1 by pulling out the piston 7, the user can inject the substance contained in the fillable area 11 by introducing the piston 7 using pressure on the actuation section 13. At the end of the injection process, the disposable syringe 1 is in the end position shown in FIG. 2 in which the latching section 19 has passed the counter-latching section 27 such that the stop surface 21 is disposed opposite of the counter-stop surface 29. The latching section 19 and the counter-latching section 27 are also in a locked state, as a result of which the piston 7 can only again be pulled out of the cylinder 3 if in addition to the tensile force for moving the piston section Fz, an additional latching force FR of the locked latching sections 19, 27 counteracting the removal of the piston 7 is applied. The introduction of such a high force into the piston 7 is, however, avoided in that the maximum force FB tolerated by the predetermined breaking point 15 is selected significantly lower than the sum of the tensile force FZ required for removing the piston section and the latching force FR. If the user tries to pull the piston 7 of the disposable syringe 1 out of the cylinder 3 a second time, the predetermined breaking point 15 breaks, thereby detaching the actuation section 13 from the remainder of the piston 7 and rendering the disposable syringe 1 unusable.

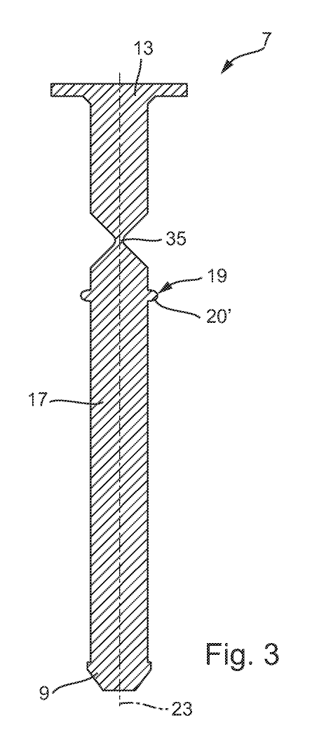

The piston 7 of the disposable syringe 1 can also be designed as shown in FIG. 3. Here the predetermined breaking point 15, instead of the transverse hole 16, has a thinned area 35, the diameter of which is dimensioned such that the predetermined breaking point 15 breaks when applying a tensile force which meets or exceeds the force FB. The latching section 19 is formed here by an annular collar 20′ having a symmetrical cross-section, wherein the surface of the annular collar 20′ is curved. As an alternative, the latching section 19 may also have an asymmetrical cross-section and/or a planar surface, at least in some sections.

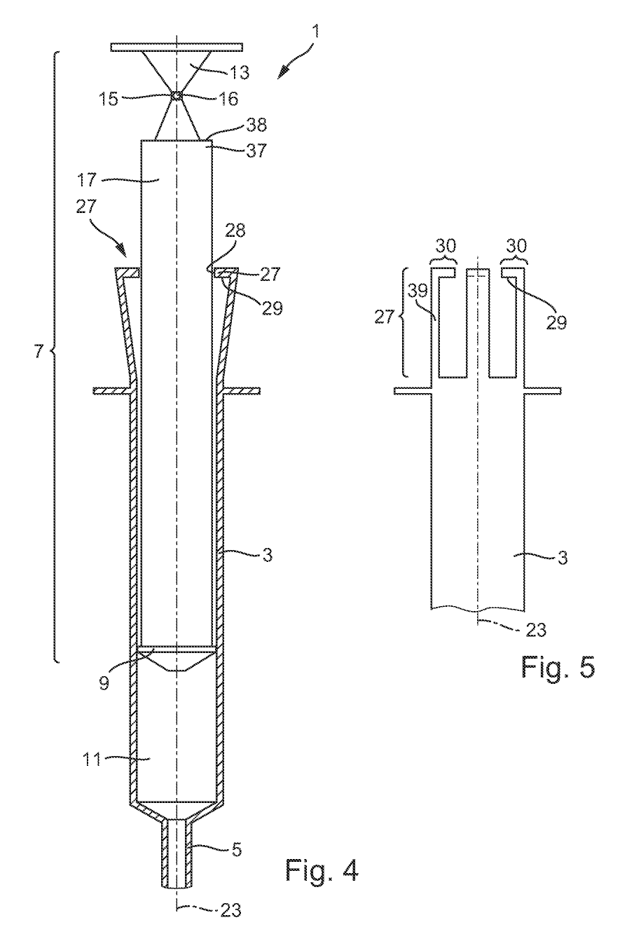

Instead of an outwardly protruding latching section 19, a latching section that is formed by a recess in the piston rod may be provided, which has a shoulder 37 that is shown in FIG. 4 and includes a surface 38 disposed orthogonally to the center longitudinal axis 23 of the piston 7. This surface 38, in contrast to the form shown in FIG. 4, can also be inclined with respect to the center longitudinal axis 23 of the piston 7 or have a curved shape deviating from the planar shape shown in FIG. 4. The counter-latching section 27 is also designed in a ring-like manner protruding inward, like the counter-latching section 27 of the form shown in FIG. 1, and delimits the narrow point 28 in the cylinder 3. Here, however, the counter-latching section 27, as long as it is not locked, has pretension, that is, the cylinder 3 is elastically expanded outward in the vicinity of the counter-latching section 27 and rests with pretension against the piston rod 17.

In the locked state, which is assumed when the shoulder 37 has passed the counter-stop surface 29 of the counter-latching section 27 upon introduction of the piston 7 into the cylinder 3, the counter-stop surface 29 of the counter-latching section 27 engages behind the shoulder 37 in that it moves radially inward under the influence of the pretension. The locking force FR then acts against any pulling on the piston 7 such that further pulling on the actuation section 13 results in breakage of the predetermined breaking point 15, just as in the form shown in FIG. 1, and thus results in destruction of the disposable syringe 1.

In a form not depicted, the cylinder 3 of the disposable syringe 1 comprises one or more sections on the counter-latching section 27 in which the wall thickness of the cylinder 3 is reduced. As a result, the elasticity of the counter-latching section 27 is increased, and a pressing force of the counter-latching section 27 against the piston rod 17 acting radially to the center longitudinal axis 23 of the piston 7 is reduced, which permits the production of an especially smooth disposable syringe 1. Instead of the areas having a reduced wall thickness, notches, recesses and the like can be provided on the cylinder 3.

Furthermore a predetermined breaking point 15 can be provided in this form, which has the thinned area 35 instead of the transverse hole 16.

The form shown in FIG. 5 shows another possibility of creating a counter-latching section 27 having higher elasticity, in which four flexible tongues 39 with inwardly protruding partial sections 30 form the latching section 27, said tongues protruding in an axial direction over the side of the cylinder 3 facing away from the injection nozzle 5. Depending on the properties of the material used to produce the disposable syringe 1 and the dimensions of the disposable syringe 1, it can be useful or necessary to provide a different number of flexible tongues 39 or to match the geometry, in particular the length, of the flexible tongues 39.

If the piston 7 with the shoulder 37 shown in FIG. 4 is placed in such a cylinder 3 of a disposable syringe 1, then the flexible tongues 39 are elastically bent outward radially to the center longitudinal axis 23 of the piston 7, as long as the latching section 19 and the counter-latching section 27 are not locked to each other. As soon as the shoulder 37 has passed the counter-stop surface 29 of the counter-latching section 27 upon introduction of the piston 7 into the cylinder 3, the partial sections 30 move radially inward due to the spring force produced by the flexible tongues 39, so that the counter-stop surface 29 of each partial section 30 engages behind the shoulder 37 and thus locks the latching section 19 and the counter-latching section 27 to each other and counteracts removal of the piston 7 from the cylinder 3.

The counter-latching section 27 in the form shown in FIG. 5 comprises several inwardly directed partial sections 30, while the counter-latching sections 27 of the forms described in FIGS. 1 to 4 comprise a single narrow point 28 surrounding the entire circumference of the cylinder 3. In the latter forms, it may also be provided, like in the form shown in FIG. 5, that the counter-latching section 27 comprises several segment-like partial sections 30 which protrude radially into the cylinder 3.

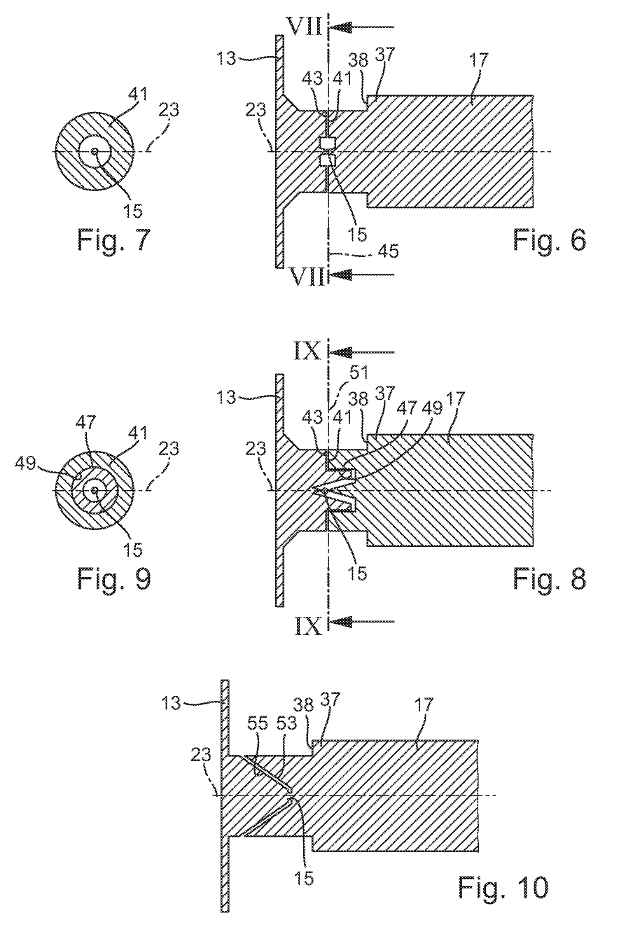

FIG. 6 shows part of the piston 7 with the piston rod 17, the predetermined breaking point 15 and the actuation section 13, wherein the piston rod 17 includes a contact section 41 orthogonal to the center longitudinal axis 23 of the piston 7 and the actuation section includes a counter-contact section 43 orthogonal to the center longitudinal axis 23 of the piston 7. As an alternative to the planar orthogonal contact section 41 and the counter-contact section 43 shown in FIG. 6, they can also have uneven surfaces. They can, for example, be serrated or grooved. When the piston 7 is introduced into the cylinder 3 by applying pressure on the actuation section 13, the orthogonal contact section 41 and the orthogonal counter-contact section 43 act against each other and thus absorb at least part of the pressure force which is needed to introduce the piston 7, and they thus prevent a bending and/or breaking of the predetermined breaking point 15 upon introduction of the piston 7 into the cylinder 3.

As illustrated in FIG. 7, which depicts a cut through the piston rod 17 shown in FIG. 6 along a cut line 45, the orthogonal contact section 41 has the shape of a disc. Accordingly, the orthogonal counter-contact section 43 is also configured in the shape of a disc. Deviating therefrom, the orthogonal contact section 41 and/or the corresponding counter-contact section 43 may have different shapes, in particular they can comprise one or more segments of a circle.

In addition, the piston rods 17 and the actuation section 13 each can include a contact section 47 longitudinally running parallel to the center longitudinal axis 23 of the piston 7 as shown in FIG. 8 and a longitudinally running counter-contact section 49. Both when introducing the piston 7 into the cylinder 3 and when removing the piston 7 from the cylinder 3, the longitudinally running contact surface 47 acts against the corresponding counter-contact section 49 in order to absorb such forces which act orthogonal to the center longitudinal axis 23 of the piston 7 in order to prevent any interfering back-and-forth movements of the actuation section 13 or even breakage of the predetermined breaking point 15 because of these forces.

FIG. 9 shows a cut along the cut line 51 through the arrangement shown in FIG. 8. Here too, the orthogonal contact section 41, the orthogonal counter-contact section 43, the longitudinally running contact section 47 and the longitudinally running counter-contact section 49 enclose the entire circumference of the piston 7, however, they may be designed such that they only enclose sections of the circumference of the piston 7, in other words, they have a segment-like design.

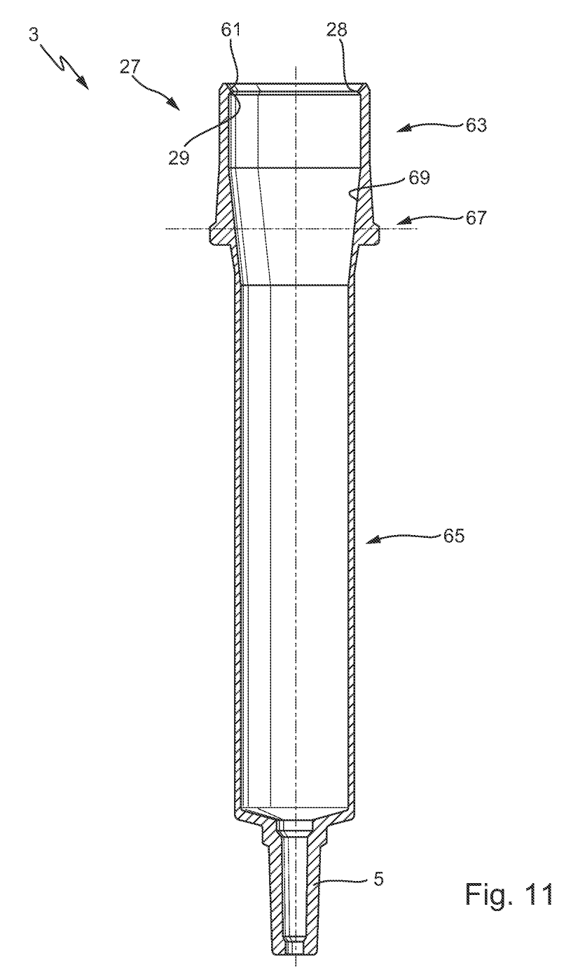

In the form shown in FIG. 10, a transversely running contact section 53 and a transversely running counter-contact section 55 are disposed obliquely to the center longitudinal axis 23 of the piston 7, wherein the transversely running contact section 53 of the actuation section 13 of the piston rod 17 is configured conically and the corresponding counter-contact section 55 has a tapered shape. Since because of the inclined position of sections 53, 55 they can absorb the pressure forces applied along the center longitudinal axis 23 of the piston 7 in order to insert the piston 7 into the cylinder 3, the form shown in FIG. 10 comprises no orthogonal contact section 41 or counter-contact section 43. Accordingly, in order to even better absorb the pressure forces, these sections may be provided in addition to the transversely running contact section 53 or the corresponding counter-contact section 55. In order to even better absorb lateral forces, the longitudinally running contact section 49 and the corresponding counter-contact section 55 can be provided in addition.

Another form of the present disclosure is depicted in FIGS. 11 to 14. The counter-latching section 27 of the cylinder 3 shown in FIG. 11 is disposed at an end of the cylinder 3 facing away from the injection nozzle 5 and is formed by an annular constriction. The resultant ring-shaped narrow point 28 surrounding the entire circumference of the cylinder 3 is bounded especially on the side thereof facing the injection nozzle 5 by the counter-stop surface 29 and on the side thereof facing away from the injection nozzle 5 by another insertion taper 61.

The cylinder 3 at the end thereof facing away from the injection nozzle 5 comprises a first area 63 with an increased inside and outside diameter. A second area 65 of the cylinder 3 facing the injection nozzle 5 has a smaller inside and outside diameter than the first area 63 of the cylinder 3. Placed between the first area 63 and the second area 65 is a transition area 67 of the cylinder 3 on which the inside diameter of the cylinder 3 continually decreases in the direction of the injection nozzle 5. The inside diameter of the cylinder can decrease linearly such that an inside surface 69 of the transition area 67 has a conical shape.

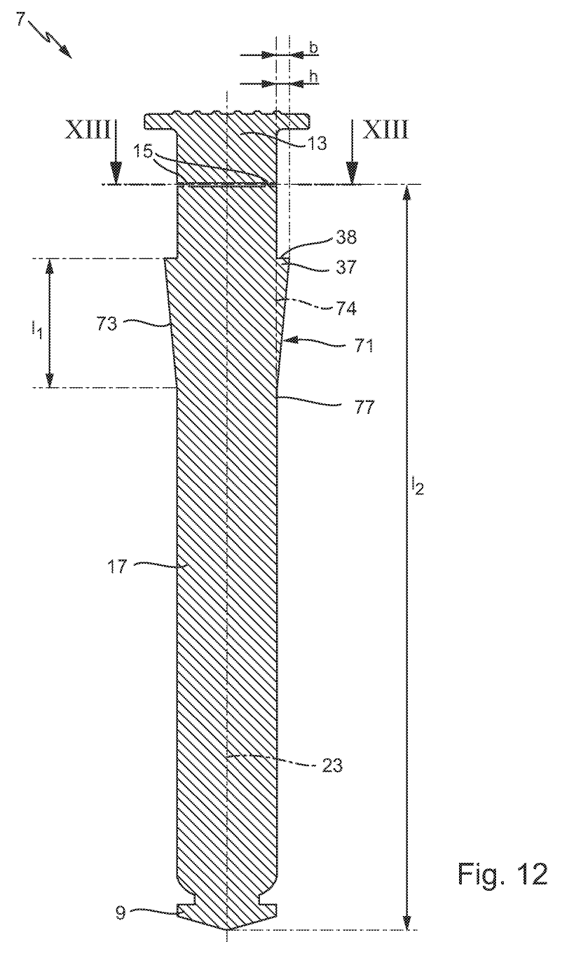

On the piston rod 17, the piston 7 shown in FIG. 12 includes an elongated chamfer 71 which is bounded at the end thereof facing the actuation section 13 by the surface 38 of the shoulder 37 disposed at least substantially orthogonal to the center longitudinal axis 23. At this chamfer 71, the outside diameter of the piston rod 17 increases continuously in the direction of the actuation section. A length l₁ of the chamfer 71 amounts at least to one-tenth of a length l₂ of the piston rod 17 including the piston section 9. In the form shown, the length l₁ of the chamfer 71 amounts to about 17 percent of length l₂. The chamfer 71 has a substantially linear course. The shape of the chamfered surface 73 of the piston rod 17 can correspond at least mostly to the inside surface 69 of the transition area 67.

It is apparent from FIG. 12 that the length l₁ of the chamfer 71 significantly exceeds the height h of the chamfer 17. The length l₁ of the chamfer corresponds approximately to ten times the height h of the chamfer 71. The chamfer 71 has an at least substantially triangular cut surface in the longitudinal cut of the piston 7. The height h of the chamfer 71 corresponds to a distance between a base 74 of the cut surface of the chamfer 71 running parallel to the center longitudinal axis 23 and the edge of the surface 38 facing away from the center longitudinal axis 23. In the form shown, the height h of the chamfer 71 corresponds to a width b of the shoulder 37.

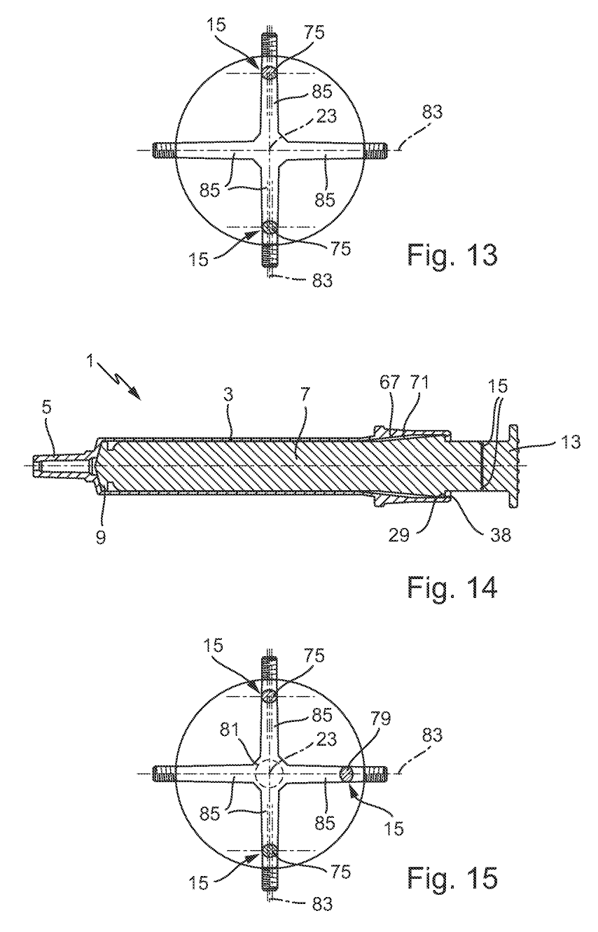

The piston 7 comprises two predetermined breaking points 15. It is apparent from FIG. 13 that each predetermined breaking point 15 is formed by a film-like connection surface 75. A distance between the two connection surfaces 75 is at least half of the outside diameters of the piston rod 17. In the form shown, the connection surfaces 75 are disposed symmetrically to the center longitudinal axis 23 on a plane orthogonal to the center longitudinal axis 23.

When introducing the piston 7 into the cylinder 3 of the disposable syringe 1, a lower end 77 of the chamfer 71 first passes the narrow point 28 of the cylinder 3. Then, depending on the exact shape and in particular the elasticity of the piston 7 and the cylinder 3, the narrow point 28 is expanded bit by bit and/or the piston rod 17 is elastically deformed at least in the area of the chamfer 71. In particular, when the piston rod, as described below, is composed of segments 85 disposed in a star-like manner, it may be provided that the piston rod or the individual segments 85 are pushed aside in a direction transverse to the longitudinal direction. In the process a pretension force builds, which acts radially against the chamfer 71 and is exerted by the narrow point 28 with respect to the chamfer 71. Because of the relatively large length l₁ of the chamfer 71, a comparatively low axial force must be exerted here on the actuation section 13. Since the outside diameter of the chamfer 71 increases continuously and slowly in the axial direction, no abrupt increase in resistance that would be perceived by the user of the disposable syringe 1 occurs during the introduction of the piston 7 into the cylinder 3, which means a force necessary to introduce the piston 7 into the cylinder 3.

Since the piston 7 of the disposable syringe 1 comprises several connection surface 75 disposed at a distance from each other, a connection formed by the connection surfaces 75 between the actuation section 13 and the piston rod 17 can also absorb laterally acting forces, in addition to the axial force needed to introduce the piston 7, thereby preventing bending of the actuvation section 13.

Once the surface 38 of the shoulder 37 has passed the narrow point 28, the inside diameter of the narrow point 28 is reduced and the counter-latching area 29 of the cylinder 3 engages behind the shoulder 37 of the piston 7 at the surface 38 (see FIG. 14).

Once the counter-latching area 29 has surrounded the shoulder 37, removal of the piston 7 from the cylinder 3 is made more difficult. An attempt to remove the piston 7 on the actuation section 13 again from the cylinder 3 would prompt the predetermined breaking points 15 at the connection surfaces 75 to break.

In another form, an additional predetermined breaking point 15 is provided in the form of another connection surface 79, as shown in FIG. 15. All three connection surfaces 75, 79 are disposed on a plane orthogonal to the center longitudinal axis 23. All connection surfaces 75, 79 have the same distance to the center longitudinal axis 23. This distance amounts to at least half of the radius of the piston rod 17 in the area of the orthogonal plane.

In addition, a center connection surface 81 can also be provided in a center area of the piston rod 17 and can also be located in the orthogonal plane. A distance between the predetermined breaking points 15 formed by the connection surfaces 75, 79, 81 in the form shown in FIG. 15 is at least half of the radius of the piston rod 17 in the area of the orthogonal plane.

It is apparent from FIGS. 13 and 15 that the piston rod 17 has a cross-shaped cross-section. The above information on the diameter and radius of the piston rod 17 relate to the longitudinal planes 83 in which segments 85 resulting from the cross-shape are located. Four segments 85 are provided in the form shown. An alternative number of segments 85 can also be provided.

It should be noted that the disclosure is not limited to the various forms described and illustrated as examples. A large variety of modifications have been described and more are part of the knowledge of the person skilled in the art. These and further modifications as well as any replacement by technical equivalents may be added to the description and figures, without leaving the scope of the protection of the disclosure and of the present patent.

What is claimed is:

1. A disposable syringe with protection against reuse, comprising a cylinder with an injection nozzle and a piston that has a piston section facing the injection nozzle, a piston rod and an actuation section facing away from the piston section, wherein the piston rod has a latching section with a shoulder that is provided in or on the piston rod and runs radially inward, the cylinder has a counter-latching section protruding inward, and the piston has at least one predetermined breaking point in such a way that when the piston rod is introduced into the cylinder the counter-latching section engages behind the shoulder to lock it and counteracts removal the piston rod from the cylinder, and wherein the predetermined breaking point is designed such that it breaks when pulling on the actuation section.

2. The disposable syringe according to claim 1, characterized in that the counter-latching section rests under pretension against the piston rod during at least most of the insertion path of the piston.

3. The disposable syringe according to claim 1, characterized in that the latching section rests under pretension against the counter-latching section during a section of the insertion path of the piston, wherein in order to produce the pretension the latching section comprises a chamfer which is bounded by the shoulder on a side facing the actuation section, whereby the chamfer has a length (l₁) extending in the axial direction that amounts at least to 1.5 times, preferably at least five times, and more preferred at least ten to twenty times the height (h) of the chamfer extending in the radial direction.

4. The disposable syringe according to claim 3, characterized in that the length (l₁) of the chamfer corresponds to at least one tenth of the length (l₂) of the piston rod with the piston section.

5. The disposable syringe according to claim 3, characterized in that the counter-latching section is provided in a cylinder section that has a larger inside diameter compared to the main cylinder section of the cylinder.

6. The disposable syringe according to claim 2, characterized in that the counter-latching section is designed to be resilient at least in some sections in the radial direction in order to produce the pretension.

7. The disposable syringe according to claim 6, characterized in that the counter-latching section comprises at least one flexible tongue, which is designed to be resilient in the radial direction and extends in the axial direction, in order to produce the pretension, said tongue having a partial section protruding radially inward and interacting with the shoulder.

8. The disposable syringe according to claim 3, characterized in that the latching section and/or the chamfer of the latching section are designed to be resilient in the radial direction and/or in a direction running transversely to the longitudinal direction in order to produce the pretension.

9. The disposable syringe according to claim 1, characterized in that the predetermined breaking point is located between the piston rod and the actuation section.

10. The disposable syringe according to claim 1, characterized in that the piston rod and/or the actuation section comprise contact sections interacting with each other and designed separately from the predetermined breaking point in such a way that bending or breaking of the predetermined breaking point is prevented when the piston is pressed in.

11. The disposable syringe according to claim 10, characterized in that the contact sections have a surface area running approximately or exactly perpendicularly and/or parallel to the center longitudinal axis of the piston.

12. The disposable syringe according to claim 10, characterized in that the contact sections of the piston rod and the actuation section are both designed conically and run at least substantially parallel to each other.

13. The disposable syringe according to claim 10, characterized in that contact sections are provided in the area around the predetermined breaking point.

14. The disposable syringe according to claim 1, characterized in that the piston has at least two predetermined breaking points designed as connection surfaces which are disposed at a distance from each other, preferably within a plane running orthogonal to the center longitudinal axis of the piston.

15. The disposable syringe according to claim 1, characterized in that the piston has a predetermined breaking point designed as a center connection surface at a center region of a cross-sectional surface of the piston rod, wherein the center connection surface is preferably disposed within the plane running orthogonal to the center longitudinal axis of the piston.

16. The disposable syringe according to claim 1, characterized in that a distance between the predetermined breaking points amounts to at least half of a radius of the piston rod.

17. The disposable syringe according to claim 1, characterized in that the piston rod and/or the actuation section at least in some sections of the cross-section are composed of several, preferably four, segments protruding radially in a star shape from the center longitudinal axis.

18. The disposable syringe according to claim 1, characterized in that the piston section, the piston rod, the actuation section and the latching section are connected to each other in a single piece and/or the cylinder and the counter-latching section are connected to each other in a single piece.
